(19) United States
(12) Patent Application Publication
(10) Pub. No.: US 2024/0139362 A1
(43) Pub. Date: May 2, 2024

(54) STERILIZATION APPARATUS

(71) Applicant: SL Corporation, Daegu (KR)

(72) Inventors: Hyun Woo Choi, Gyeongsan-si (KR); Sang Hyoung Lee, Gyeongsan-si (KR); Jong Min Lee, Gyeongsan-si (KR); Jong Woon Kim, Gyeongsan-si (KR); Hae Ryun Lee, Gyeongsan-si (KR); Man Young Chun, Gyeongsan-si (KR); Hye Jin Park, Gyeongsan-si (KR)

(21) Appl. No.: 18/410,967

(22) Filed: Jan. 11, 2024

Related U.S. Application Data
(63) Continuation of application No. PCT/KR2022/005025, filed on Apr. 7, 2022.

(30) Foreign Application Priority Data
Jul. 22, 2021 (KR) 10-2021-0096424

(51) Int. Cl. A61L 2/10; A61L 2/26

(52) U.S. Cl. CPC A61L 2/10; A61L 2/26; A61L 2202/11; A61L 2202/121; A61L 2202/122

(57) ABSTRACT

A sterilization apparatus includes a housing; a sterilization unit for irradiating, with sterilization light, at least one region to be sterilized; and a driving unit for moving the sterilization unit into and out of the housing via an opening of the housing and rotating the sterilization unit about a rotation axis.

### CROSS-REFERENCE TO RELATED APPLICATIONS

This application is a Continuation of International Application No. PCT/KR2022/005025 filed Apr. 7, 2022, which claims priority from Korean Application No. KR 10-2021-0096424 filed Jul. 22, 2021. The aforementioned applications are incorporated herein by reference in their entireties.

### TECHNICAL FIELD

The present disclosure relates to a sterilization apparatus, and more particularly, to a sterilization apparatus capable of facilitating sterilization for a variety of sterilization target areas.

### RELATED ART

Typically, public transportation, such as buses and taxis, or multi-purpose facilities, such as department stores and discount stores, involve frequent hand contact by numerous individuals. In cases where various germs and foreign substances adhere to such contact surfaces, there is a significant risk of rapid transmission thereof to many people. Thus, meticulous management of hygiene is essential.

To address this issue, efforts have been made to disinfect areas frequently touched by people using chemicals. However, chemical sterilization results in continuous consumption of chemicals, leading to increased costs. Accordingly, sterilization using light, such as ultraviolet (UV) light, is recently being used instead of chemicals to disinfect contact surfaces.

When using light for sterilization, it is necessary to ensure that the sterilization light is applied to the entire sterilization target area. In this case, multiple light sources need to be provided.

Furthermore, when there are multiple sterilization target areas, it is required to install separate light sources for the application of the sterilization light in each area. This imposes limitations on achieving sterilization for various target areas. Additionally, reducing the number of light sources can lead to an increase in the required sterilization time to satisfy the sterilization efficiency.

Therefore, there is a demand for a solution that allows easy sterilization of various target areas with a simple setup while reducing the required sterilization time to meet sterilization efficiency.

### SUMMARY

Aspects of the present disclosure provide a sterilization apparatus capable of enabling sterilization for a variety of sterilization target areas.

However, aspects of the present disclosure are not restricted to those set forth herein. The above and other aspects of the present disclosure will become more apparent to one of ordinary skill in the art to which the present disclosure pertains by referencing the detailed description of the present disclosure given below.

A sterilization apparatus according to an embodiment of the present disclosure may include a housing; a sterilization unit that irradiates sterilization light to at least one sterilization target area; and a driving unit configured to move the sterilization unit into and out of the housing via an opening of the housing and to rotate the sterilization unit about a rotation axis.

The sterilization unit may be moved in a vertical direction through the opening, which is formed on a top surface of the housing. The sterilization unit may include at least one light source that generates the sterilization light and a mounting bracket in which the at least one light source is mounted. The sterilization unit may further include at least one reflector that reflects the sterilization light generated from the at least one light source to the at least one sterilization target area. The at least one reflector may include a plurality of reflective surfaces formed at different locations with respect to the at least one light source.

The driving unit may include a first operating part that is coupled to the sterilization unit and allows the sterilization unit to rotate about the rotation axis and a second operating part that allows the sterilization unit to move through the opening of the housing. The second operating part may allow the sterilization unit to move in a direction parallel to the rotation axis.

The second operating part may include a support bracket that supports the first operating part, and as the support bracket moves, the sterilization unit coupled to the first operating part may move through the opening of the housing. The support bracket may include at least one guide rib movably inserted in at least one guide rail, which is formed in the housing. The sterilization apparatus may further include a pressurizing member elastically deformed by the at least one guide rib. A first end portion of the pressurizing member may be coupled to the housing, and a second end portion of the pressurizing member may be elastically deformed by the at least one guide rib to exert pressure on one side of the at least one guide rib to allow the at least one guide rib to come into contact with an inner surface of the at least one guide rail. The sterilization apparatus may further include an elastic part formed to extend from the at least one guide rib, and an end portion of the elastic part may be elastically deformed by the at least one guide rail.

The sterilization apparatus may further include a cover part provided on a side of the sterilization unit. The cover part may be formed of a light-transmitting material, and an optical pattern that reflects or scatters light incident from the outside may be formed on at least one surface of the cover part.

The sterilization light may have a wavelength in a UV-C range. For example, the wavelength of the sterilization light may be between about 222 nm and about 280 nm.

Other specific details of the present disclosure are included in the detailed description and drawings.

The aforementioned sterilization apparatus of the present disclosure may provide one or more effects as follows.

As the irradiation direction of sterilization light can be varied due to the rotation of the sterilization unit, various target areas may be sterilized more easily.

Furthermore, when sterilization is needed, the sterilization unit may be exposed outside the housing, and when sterilization is not needed, the sterilization unit may be withdrawn inside the housing. This results in the effect of reducing the space occupied by the sterilization unit when sterilization is not required, thereby expanding the interior space of the vehicle.

It should be noted that the effects of the present disclosure are not limited to those described above, and other effects of the present disclosure will be apparent from the following description.

### BRIEF DESCRIPTION OF DRAWINGS

FIG. 1 is a perspective view illustrating the exterior of a sterilization apparatus according to an embodiment of the present disclosure;

FIG. 2 is a side view illustrating the exterior of the sterilization apparatus according to an embodiment of the present disclosure;

FIG. 3 is a perspective view of the sterilization apparatus according to an embodiment of the present disclosure;

FIG. 4 is a side view of the sterilization apparatus according to an embodiment of the present disclosure;

FIG. 5 is an exploded perspective view of the sterilization apparatus according to an embodiment of the present disclosure;

FIG. 6 is a perspective view of a housing according to an embodiment of the present disclosure;

FIG. 7 is a side view of a driving unit according to an embodiment of the present disclosure;

FIG. 8 is a perspective view of a support bracket movably coupled to the housing according to an embodiment of the present disclosure;

FIG. 9 is a cross-sectional view of a pressurizing member according to an embodiment of the present disclosure;

FIG. 10 is a cross-sectional view of an elastic part formed in the support bracket according to an embodiment of the present disclosure;

FIG. 11 is a side view of a sterilization unit moved upward during sterilization according to an embodiment of the present disclosure;

FIG. 12 is a side view of the sterilization unit positioned to be exposable on the outside of the housing during sterilization according to an embodiment of the present disclosure;

FIG. 13 is a perspective view of a cover part according to an embodiment of the present disclosure;

FIG. 14 is a block diagram of a sterilization system according to an embodiment of the present disclosure; and

FIG. 15 is a flowchart illustrating an operating method of the sterilization system according to an embodiment of the present disclosure.

### DETAILED DESCRIPTION

The advantages and features of the present disclosure, as well as methods for achieving them, will become clear with reference to the detailed examples provided in the accompanying drawings. However, it should be noted that the present disclosure is not limited to the embodiments disclosed herein but can be implemented in various other forms. These embodiments are provided to ensure full disclosure of the present disclosure to those skilled in the art to which it pertains, and the disclosure is defined solely by the scope of the claims. Throughout the entire specification, the same reference numerals denote the same components.

Therefore, in some embodiments, well-known process steps, well-known structures, and well-known technologies are not specifically described to avoid any ambiguity in the interpretation of the present disclosure.

The terminology used in this specification is for describing the embodiments and is not intended to limit the present disclosure. In this specification, unless otherwise specified, the singular form includes the plural form. The terms “comprises” and/or “comprising” used in the specification are intended to indicate that the named components, steps, operations, and/or elements do not exclude the presence or addition of one or more other components, steps, operations, and/or elements beyond those mentioned. Moreover, “and/or” encompasses each of the mentioned items and all possible combinations thereof.

Furthermore, the embodiments described in this specification will be explained with reference to exemplary sectional and/or schematic views of the present disclosure. Therefore, the form of the embodiments of the present disclosure may be altered by manufacturing techniques and/or permissible tolerances, among other factors. Hence, the embodiments of the present disclosure include variations in shape that may be generated according to the manufacturing process, and it should be noted that the components in each drawing of the present disclosure may be slightly enlarged or reduced for the convenience of explanation. Throughout the entire specification, the same reference numerals denote the same components.

A sterilization apparatus according to an embodiment of the present disclosure will hereinafter be described with reference to the accompanying drawings.

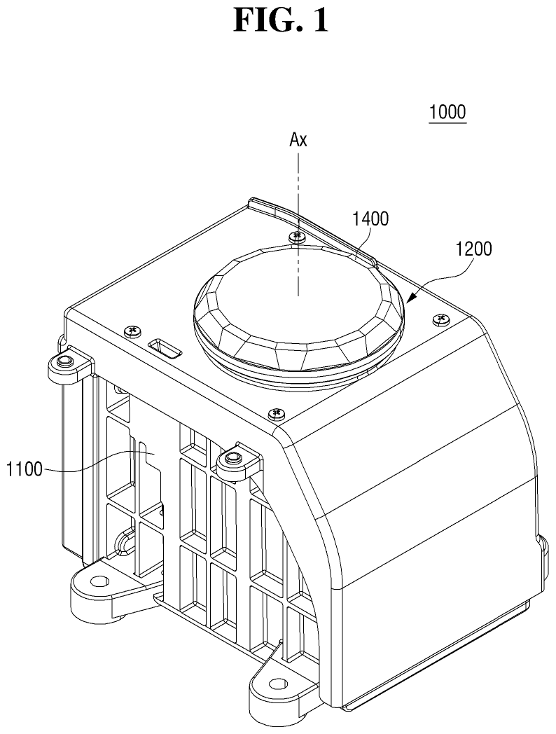

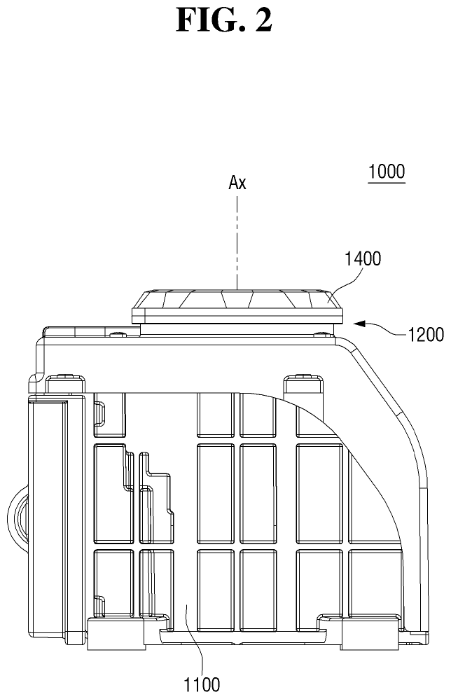

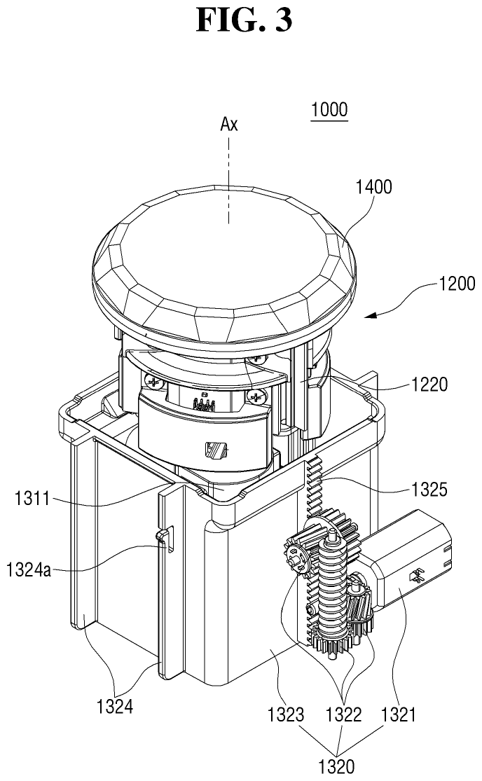

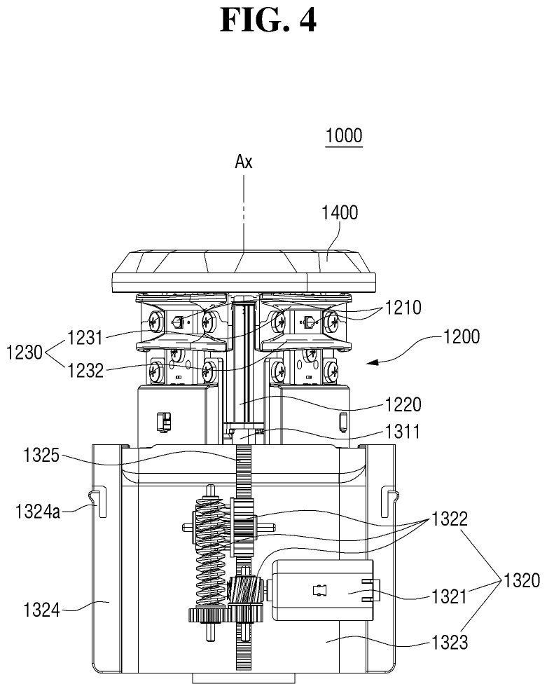

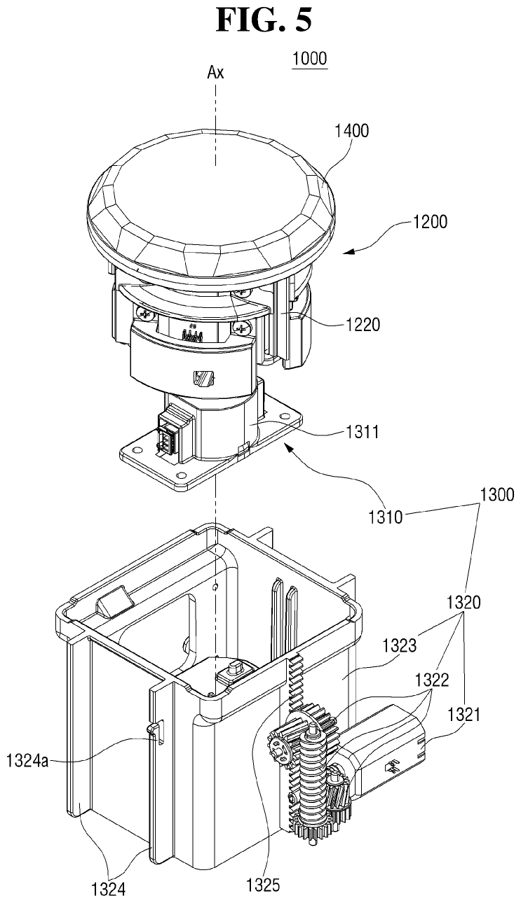

FIG. 1 is a perspective view illustrating the exterior of a sterilization apparatus according to an embodiment of the present disclosure, FIG. 2 is a side view illustrating the exterior of the sterilization apparatus according to an embodiment of the present disclosure, FIG. 3 is a perspective view of the sterilization apparatus according to an embodiment of the present disclosure, FIG. 4 is a side view of the sterilization apparatus according to an embodiment of the present disclosure, and FIG. 5 is an exploded perspective view of the sterilization apparatus according to an embodiment of the present disclosure.

Referring to FIGS. 1 through 5, a sterilization apparatus 1000 may include a housing 1100, a sterilization unit 1200, and a driving unit 1300.

The sterilization apparatus 1000 is used to prevent the spread of various infectious diseases through sterilization of public transportation means, such as buses, taxis, etc., as well as multipurpose facilities, such as buildings, vehicles, elevators, escalators, etc., thereby allowing for safe and clean utilization. In an embodiment of the present disclosure, for example, the sterilization apparatus 1000 may be installed inside a vehicle, to perform sterilization for the interior space of the vehicle.

In an embodiment of the present disclosure, for example, the sterilization apparatus 1000 may be installed near the driver's seat, such as in the center console area between the driver's seat and the passenger seat, to enable irradiation of sterilization light in the direction of the dashboard, which can be more heavily contaminated compared to other areas due to frequent contact by the driver. However, the present disclosure is not limited to the dashboard sterilization. The location where the sterilization apparatus 1000 is installed may be varied depending on the location of sterilization target areas within the vehicle's interior space.

Furthermore, in an embodiment of the present disclosure, the sterilization apparatus 1000 may be configured as, for example, a popup type that is configured to be moved upward to expose at least a portion of the sterilization unit 1200 outside the housing 1100, when performing sterilization of the vehicle's interior space, and may be configured to be moved downward to dispose at least a portion of the sterilization unit 1200 inside the housing 1100 when it is not in use. However, the present disclosure is not limited to such a configuration. The actual direction in which the sterilization unit 1200 moves for sterilizing the vehicle's interior space may vary depending on the installation location of the sterilization apparatus 1000.

The housing 1100 may include a receiving space therein to accommodate at least a portion of the sterilization unit 1200, and in an embodiment of the present disclosure, the housing 1100 may, for example, accommodate both the sterilization unit 1200 and the driving unit 1300.

In an embodiment of the present disclosure, for example, as the sterilization apparatus 1000 may be installed in the center console area, the housing 1100 may be understood as forming a part of the center console area.

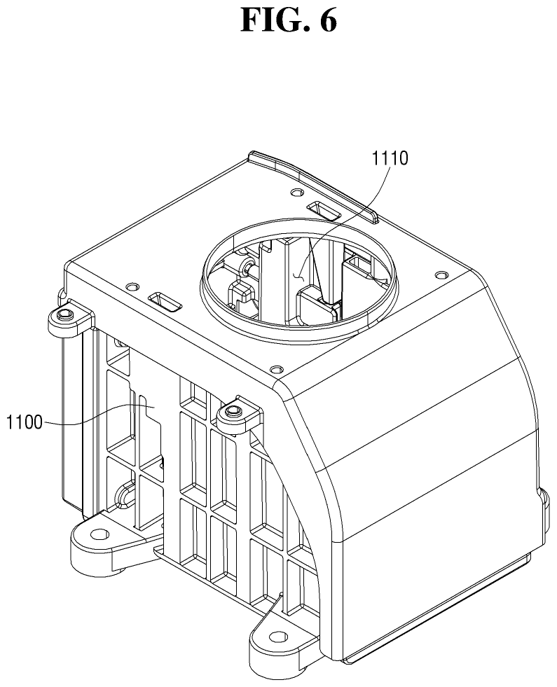

Referring to FIG. 6, the housing 1100 may have an opening 1110 that allows at least a portion of the sterilization unit 1200 to be movable between the inside and outside of the housing 1100. In an embodiment of the present disclosure, for example, as the sterilization unit 1200 may be moved in a vertical direction, the opening 1110 may be understood as being formed on the top surface of the housing 1100. However, the present disclosure is not limited to such a configuration, and the location where the opening 1110 is formed may vary depending primarily on the direction of movement of the sterilization unit 1200.

Allowing the sterilization unit 1200 to move between the inside and outside of the housing 1100 through the opening 1110 is to dispose the sterilization unit 1200 outside the housing 1100 when sterilization by the sterilization unit 1200 is needed, and to dispose the sterilization unit 1200 inside the housing 1100 and thereby secure the interior space (e.g., cabin space) of the vehicle when sterilization is not required for the vehicle's interior space.

The sterilization unit 1200 may apply sterilization light to at least one sterilization target area. As such, the sterilization unit 1200 may include at least one light source 1210 and a mounting bracket 1220.

The at least one light source 1210 may generate sterilization light with a suitable wavelength and/or intensity for the sterilization function. In an embodiment of the present disclosure, for example, a semiconductor light-emitting element such as a light emitting diode (LED) that emits UV light may be used as the at least one light source 1210, but the present disclosure is not limited thereto. In some such embodiments, the UV light may have a wavelength of about 100 nm to about 400 nm. For example, the UV light may be a short-wave UV (i.e., UV-C), which has a wavelength of about 180 nm to about 280 nm, about 200 nm to about 280 nm, or about 222 nm to about 280 nm. The wavelength or intensity of sterilization light generated from the at least one light source 1210 may vary depending on factors such as sterilization duration or sterilization efficiency.

In an embodiment of the present disclosure, for example, the at least one light source 1210 may include multiple light sources 1210, which are mounted along the perimeter of the mounting bracket 1220 at regular or varying intervals, but the present disclosure is not limited thereto. The number and spacing of the at least one light source 1210 may be varied depending on factors such as the number and location of sterilization target areas where sterilization is to be performed by the sterilization unit 1200.

Furthermore, the sterilization unit 1200 may include at least one reflector 1230 that ensures that the at least one light source 1210 emits sterilization light with the appropriate intensity and has an appropriate light irradiation area depending on the size of each sterilization target area. In an embodiment of the present disclosure, for example, as the at least one light source 1210 may include multiple light sources 1210, the at least one reflector 1230 may also include multiple reflectors 1230 corresponding to the respective light sources 1210, but the present disclosure is not limited thereto. Alternatively, one reflector 1230 may be used for multiple light sources 1210, or multiple reflectors 1230 may be used for one light source 1210.

In this case, the at least one reflector 1230 may have at least one reflective surface (1231 and 1232). In an embodiment of the present disclosure, for example, the at least one reflective surface (1231 and 1232) may include multiple reflective surfaces 1231 and 1232, which are disposed respectively above and below the corresponding light source 1210 among the multiple light sources 1210, but the present disclosure is not limited thereto. The number, locations, and sizes of reflective surfaces may vary depending on factors such as the location and size of each sterilization target area.

The mounting bracket 1220 may be configured to be rotated about a rotation axis Ax and/or moved in the direction of the rotation axis Ax by the driving unit 1300 that will be described later below. In an embodiment of the present disclosure, for example, the mounting bracket 1220 may be rotated about the rotation axis Ax, which is parallel to the vertical direction, but the present disclosure is not limited thereto. The rotation axis Ax of the mounting bracket 1220 may be formed in parallel not only to the vertical direction, but also to the direction of movement of the sterilization unit 1200.

As the mounting bracket 1220 rotates about the rotation axis Ax, the direction of sterilization light emitted from multiple light sources 1210 mounted on the mounting bracket 1220 in a circumferential direction around the rotation axis Ax may be adjusted depending on the rotation angle of the mounting bracket 1220. As a result, more sterilization target areas may be sterilized using a fewer number of light sources.

The driving unit 1300 may include a first operating part 1310 and a second operating part 1320. The first operating part 1310 may enable the sterilization unit 1200 to rotate about the rotation axis Ax, and the second operating part 1320 may enable the sterilization unit 1200 to linearly move through the opening 1110 of the housing 1100.

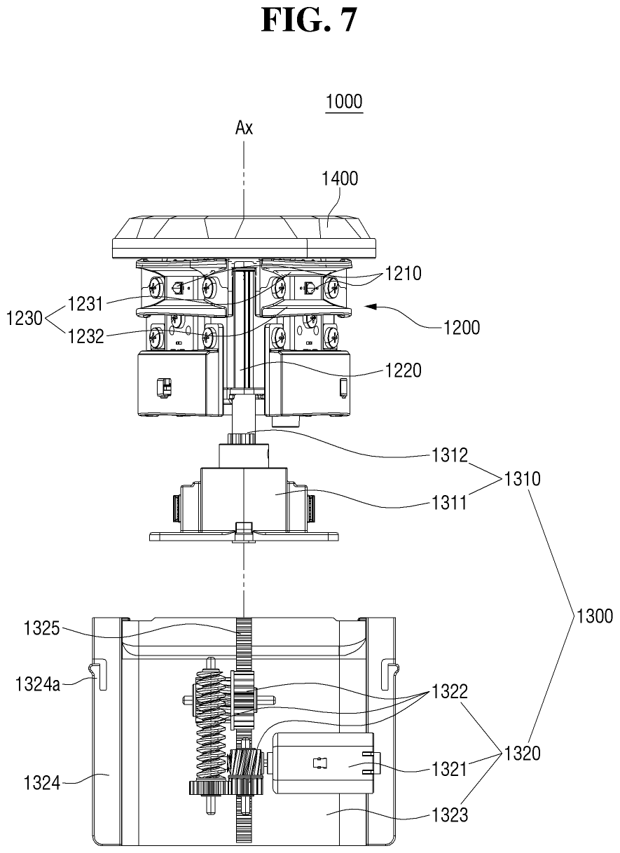

The first operating part 1310 may include a first actuator 1311 and a rotation shaft 1312. Referring to FIG. 7, the rotation shaft 1312 may be rotated about the rotation axis Ax by the first actuator 1311, allowing the sterilization unit 1200 coupled to the rotation shaft 1312 to be rotated about the rotation axis Ax.

In this case, the coupling of the rotation shaft 1312 with the sterilization unit 1200 may be understood as the rotation shaft 1312 being coupled to the mounting bracket 1220. When the mounting bracket 1220 is rotated by the rotation shaft 1312, at least one light source 1210 mounted on the mounting bracket 1220 may be rotated about the rotation axis Ax, thereby changing the direction of sterilization light emitted from the at least one light source 1210.

The second operating part 1320 may include a second actuator 1321, a gear part 1322, and a support bracket 1323. The second actuator 1321 may generate power for enabling the sterilization unit 1200 to move through the opening 1110 of the housing 1100, and the power of the second actuator 1321 may be transmitted to the support bracket 1323 via the gear part 1322, allowing the support bracket 1323 to move in a direction corresponding to the direction of movement of the sterilization unit 1200.

In an embodiment of the present disclosure, for example, the sterilization unit 1200 may move in the direction of the rotation axis Ax to be disposed outside or inside the housing 1100. In this case, the support bracket 1323 may move along the direction of the rotation axis Ax of the sterilization unit 1200. However, the direction in which the sterilization unit 1200 moves through the opening 1110 of the housing 1100 is not limited to the direction of the rotation axis Ax of the sterilization unit 1200, and the rotation axis Ax of the sterilization unit 1200 and the direction in which the sterilization unit 1200 moves through the opening 1110 of the housing 1100 may differ.

In an embodiment of the present disclosure, for example, the support bracket 1323 may support the first actuator 1311 of the first operating part 1310. As a result, as the support bracket 1323 moves in the direction of the rotation axis Ax, the sterilization unit 1200 that is coupled to the support bracket 1323 may be moved in the direction of the rotation axis Ax to be disposed outside or inside the housing 1100.

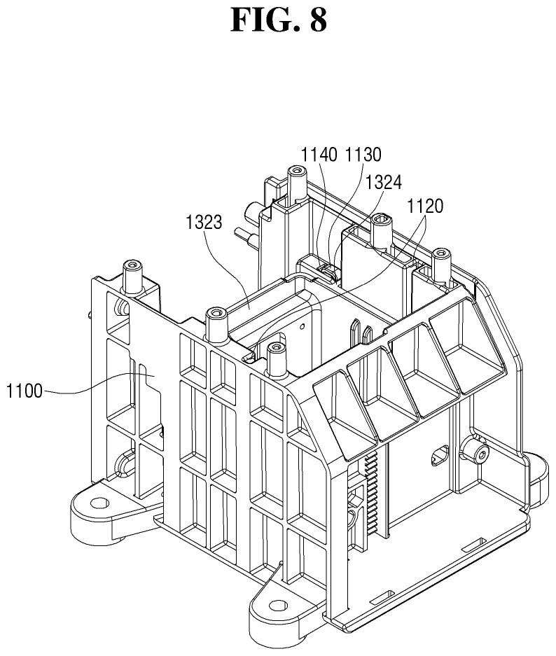

FIG. 8 is a perspective view of a support bracket that is movably coupled to a housing according to an embodiment of the present disclosure. Specifically, FIG. 8 illustrates a housing 1100 according to an embodiment of the present disclosure, with a portion thereof omitted to show the interior of the housing 1100.

Referring to FIG. 8, the support bracket 1323 may include at least one guide rib 1324 movably inserted into at least one guide rail 1120, which is formed on the inside of the housing 1100 to extend in the movement direction of the sterilization unit 1200. As the at least one guide rib 1324 moves along the at least one guide rail 1120, the movement of the support bracket 1324 may be guided.

If a gap is present in at least one direction between the at least one guide rib 1324 and the at least one guide rail 1120 when the at least one guide rib 1324 moves along the at least one guide rail 1120, unnecessary movement may occur as the sterilization unit 1200 moves. Additionally, the sterilization unit 1200 may descend at a faster rate than the ascending rate, due to the load of the sterilization unit 1200. In this case, even if the same power is transmitted from the driving unit 1300, the movement distance of the sterilization unit 1200 may vary between ascension and descension, making it difficult to accurately control the position of the sterilization unit 1200.

In an embodiment of the present disclosure, for example, a pressurizing member 1130 may be provided to apply pressure to the at least one guide rib 1324 such that the at least one guide rib 1324 and the at least one guide rail 1120 may come into contact. This configuration prevents unnecessary clearance between the at least one guide rib 1324 and the at least one guide rail 1120 in a lateral direction with respect to the movement direction of the sterilization unit 1200 and ensures that the sterilization unit 1200 has a consistent travel distance during its ascent and descent.

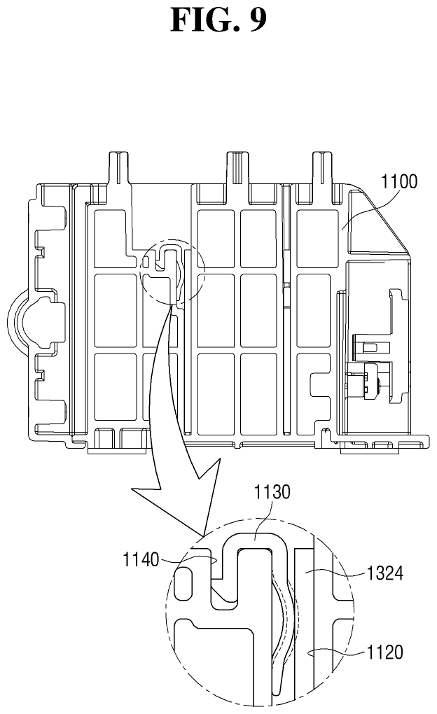

FIG. 9 is a cross-sectional view of a pressurizing member according to an embodiment of the present disclosure. Referring to FIG. 9, a first end portion of the pressurizing member 1130 may be inserted in and coupled to a coupling groove 1140 formed in the housing 1100, and a second end portion of the pressurizing member 1130 may be elastically deformed by the contact with the at least one guide rib 1324. Accordingly, both ends of the at least one guide rib 1324 may be disposed at a predetermined distance in the lateral direction with respect to the rotation axis Ax.

In an embodiment of the present disclosure, for example, both end portions of the pressurizing member 1130 may be bent in a substantially “U” shape, but the present disclosure is not limited thereto. The pressurizing member 1130 may have various shapes that can be elastically deformed by the at least one guide rib 1324 and generate a corresponding restorative force.

Thus, when the second end portion of the pressurizing member 1130 is elastically deformed by the at least one guide rib 1324, the restorative force may cause the at least one guide rib 1324 to come into contact with the inner surface of the at least one guide rail 1120. Accordingly, clearance may be prevented or minimized.

Additionally, an elastic part 1324a, which is disposed to have an end portion thereof in contact with the inner surface of the at least one guide rail 1120, may be formed in the at least one guide rib 1324.

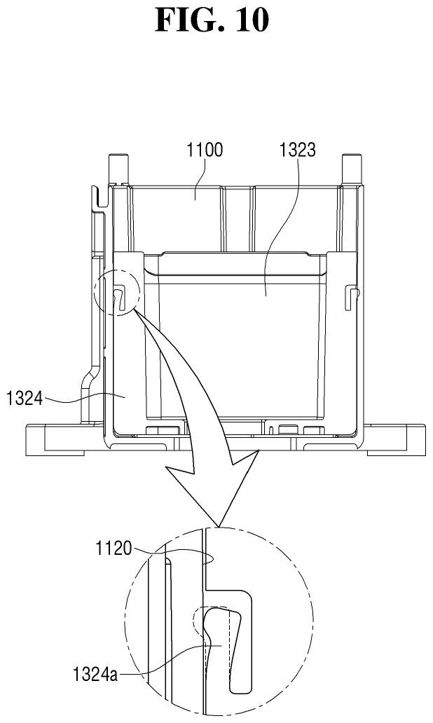

FIG. 10 is a cross-sectional view of the elastic part formed in the support bracket according to an embodiment of the present disclosure. Referring to FIG. 10, the elastic part 1324a may be formed in the guide rib 1324 of the support bracket 1323 to be elastically deformed by the force from the inner surface of the at least one guide rail 1120. When the elastic part 1324a is elastically deformed, unnecessary clearance may be prevented between the at least one guide rib 1324 and the at least one guide rail 1120 in the lateral direction with respect to the movement direction of the sterilization unit 1200 due to the restorative force of the elastic part 1324a.

In an embodiment of the present disclosure, for example, the at least one guide rib 1324 and the at least one guide rail 1120 may be formed on opposite sides of the support bracket 1323. In this case, the elastic part 1324a may be formed in each of the guide ribs 1324 on opposite sides of the support bracket 1323.

In the above-described embodiments, the pressurizing member 1130 may be used to prevent clearance in a first direction perpendicular to the movement direction of the support bracket 1323, and the elastic part 1324a may be used to prevent clearance in a second direction perpendicular to the movement direction of the support bracket 1323 and to the first direction. However, the present disclosure is not limited to such a configuration. Either the pressurizing member 1130 or the elastic part 1324a may be used for any of the first and second directions.

For example, in the support bracket 1323, rack gears 1325 may be formed along the movement direction of the sterilization unit 1200. In this example, the gear part 1322 may include one or more pinion gears, allowing the support bracket 1323 to move vertically due to the power of the second actuator 1321.

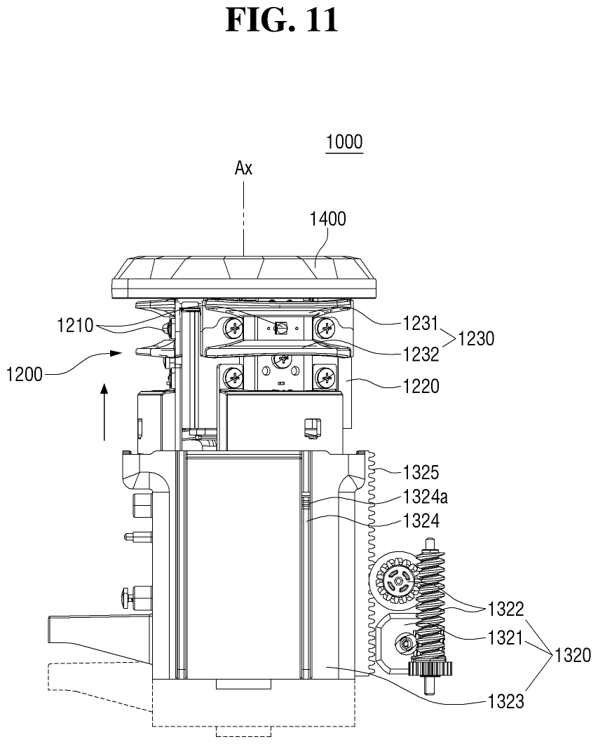

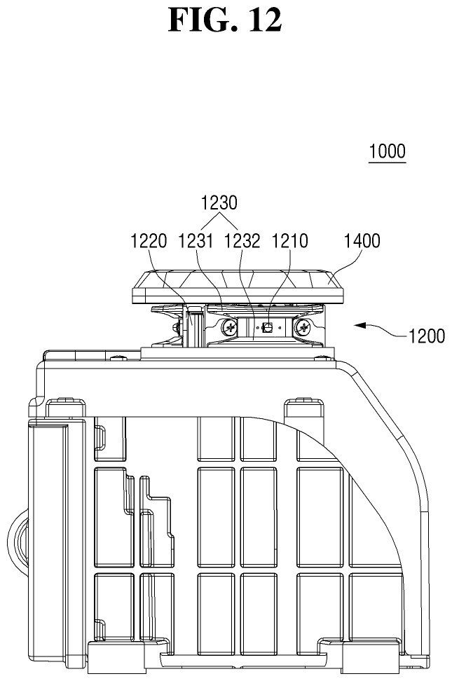

In the above-described sterilization apparatus 1000, when sterilization is needed, the support bracket 1323 may be moved upward by the second operating part 1320, as illustrated in FIG. 11. As a result, as illustrated in FIG. 12, the sterilization unit 1200 may be exposed outside the housing 1100 for irradiating the sterilization light. Conversely, when sterilization is not needed, the support bracket 1323 may be moved downward by the second operating part 1320. As a result, as illustrated in FIG. 2, the sterilization unit 1200 may be withdrawn inside the housing 1100, ensuring that the sterilization unit 1200 does not protrude into the interior space of the vehicle when no sterilization is needed.

Meanwhile, the sterilization apparatus 1000 may further include a cover part 1400, which is provided in the sterilization unit 1200 to prevent foreign substances from entering from the outside by covering the opening 1110 of the housing 1100 when the sterilization unit 1200 is withdrawn into the housing 1100, and to enhance the exterior appearance.

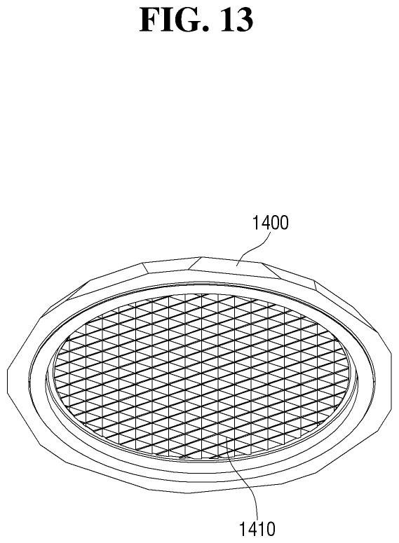

FIG. 13 is a perspective view of the cover part according to an embodiment of the present disclosure. Referring to FIG. 13, in an embodiment of the present disclosure, the cover part 1400 may be formed of a transparent material that allows light to pass through, and an optical pattern 1410 may be formed on at least one surface of the cover part 1400. Light that is incident from the outside may be reflected or scattered by the optical pattern 1410, thereby creating an image such as a glittering image.

In an embodiment of the present disclosure, for example, the optical pattern 1410 may be formed on a surface of the cover part 1400 that faces the sterilization unit 1200. However, the location and shape of the optical pattern 1410 may vary depending on a desired image to be implemented through the cover part 1400.

A sterilization operation by the sterilization apparatus 1000 may be controlled by a sterilization system that determines at least one sterilization condition and controls the sterilization apparatus 1000 based on the results of the determination.

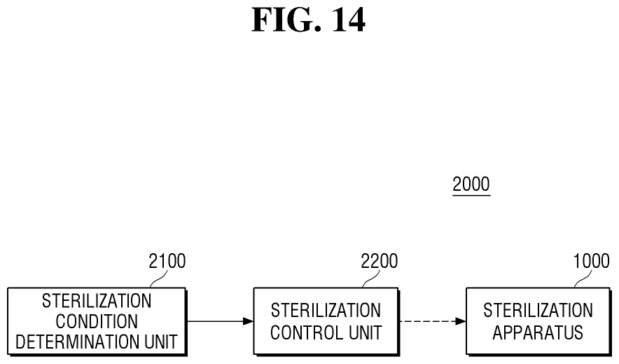

FIG. 14 is a block diagram of a sterilization system according to an embodiment of the present disclosure. Referring to FIG. 14, a sterilization system 2000 may include a sterilization condition determination unit 2100 and a sterilization control unit 2200.

The sterilization condition determination unit 2100 may be configured to determine at least one sterilization condition required for sterilization to be performed.

In an embodiment of the present disclosure, the sterilization condition determination unit 2100 may be configured to determine various sterilization conditions such as ignition status, door open/close status, passenger presence, battery level, temperature, or the like, and may be equipped with or operationally connected with sensors to detect these various sterilization conditions.

The sterilization condition determination unit 2100 may be configured to output an operation signal to the sterilization control unit 2200 when the multiple sterilization conditions are satisfied simultaneously. If sterilization is performed when at least one of the multiple sterilization conditions is not met, it may have adverse effects on humans or increase the risk of accidents during the vehicle operation.

For example, the sterilization condition determination unit 2100 may be configured to output the operation signal only when conditions such as the vehicle's ignition being off, no passenger present, doors closed, battery level above a threshold, and temperature within an acceptable range are met. This is to prevent sterilization from being initiated when the vehicle's ignition is on or doors are open, even in the absence of passengers, since there is a likelihood of passengers re-entering the vehicle in such circumstances. Similarly, if the battery level is below the threshold, it may result in issues such as the vehicle being unable to be started, and thus, in such cases, sterilization may be prevented.

Additionally, when the temperature around the space where sterilization is to be performed is outside an acceptable range, issues such as malfunction that may be caused by overheating or failure to satisfy sterilization efficiency or duration requirements may arise. Thus, the sterilization condition determination unit 2100 may ensure that the operation signal is output only when all of the aforementioned sterilization conditions are met concurrently.

The sterilization conditions determined by the sterilization condition determination unit 2100 are not limited to those set forth herein, and may be added, removed, or modified to ensure that no adverse effects are incurred on humans and no problems are caused during the vehicle operation.

When the operation signal is output from the sterilization condition determination unit 2100, the sterilization control unit 2200 may be configured to control the sterilization apparatus 1000 to ensure that sterilization light is irradiated onto at least one sterilization target area.

For example, when the operation signal is output from the sterilization condition determination unit 2100, the sterilization control unit 2200 may be configured to supply power to the second operating part 1320 to move the support bracket 1323 upward and thereby to expose the sterilization unit 1200 to the outside of the housing 1100. Subsequently, electric power may be supplied to the at least one light source 1210 to generate the sterilization light and at the same time to the first operating part 1310 to allow the sterilization unit 1200 to rotate around the rotation axis Ax.

The sterilization control unit 2200 may be configured to perform sterilization according to predefined settings when the operation signal is output from the sterilization condition determination unit 2100.

For example, the predefined settings values may include the intensity of sterilization light to be irradiated from the sterilization unit 1200, the vertical position of the sterilization unit 1200, the rotational speed of the sterilization unit 1200, or the like. The driver or user may set such settings in advance via input devices such as switches, buttons, touch screens, or the like installed in the vehicle. The sterilization control unit 2200 ensures that sterilization is performed according to the predefined settings.

Moreover, the driver may configure the settings in advance for different temperature settings, considering that sterilization efficiency and duration may vary with temperature, and the sterilization control unit 2200 may enable sterilization based on predefined settings for each detected temperature.

The sterilization control unit 2200 may be configured to continuously receive the results of the determination of the sterilization conditions from the sterilization condition determination unit 2100 during the sterilization. If the temperature is outside the acceptable range or the battery level drops below the threshold while sterilization is underway, the sterilization may be stopped or the rotation speed of the sterilization unit 1200 may be reduced to prevent malfunction from overheating and avoid adverse effects during the vehicle operation.

The results of sterilization performed by the sterilization system 2000 may be displayed through a display device provided in the sterilization apparatus 1000. The driver may use the display device to check the status of sterilization and the rate of progress in sterilization.

For example, in an embodiments of the present disclosure, the display device may be installed on one side of the sterilization apparatus 1000, for example, the top side of the sterilization apparatus 1000, but the present disclosure is not limited thereto. The display device may be installed not only in the sterilization apparatus 1000, but also at various other locations that can be easily identified by the driver.

In some embodiments, the driver may adjust the predetermined settings and control the sterilization system 2000 via a mobile app executed on a mobile device. In some such embodiments, the mobile device may function as the display device to display the sterilization results. The mobile device may also function as the input device to allow the driver or the user to adjust the settings. In some embodiments, the sterilization condition determination unit 2100 and/or the sterilization control unit 2200 may be provided as software modules of the mobile app, and the mobile device that runs the mobile app may be directly or indirectly connected to the sterilization apparatus 1000 wirelessly or otherwise.

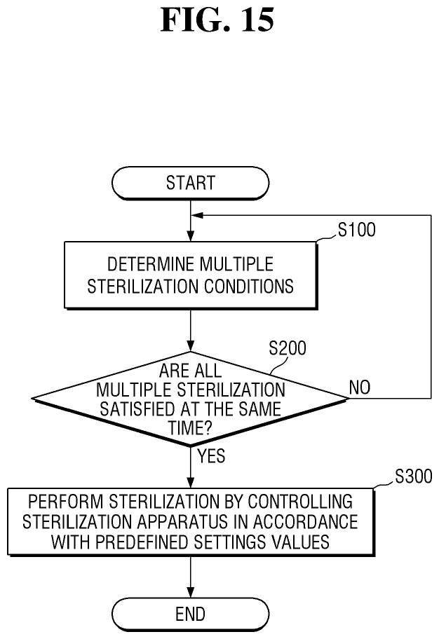

FIG. 15 is a flowchart illustrating an operating method of a sterilization system according to an embodiment of the present disclosure. FIG. 15 depicts an example in which the sterilization condition determination unit 2100 is configured to determine multiple sterilization conditions.

Referring to FIG. 15, the operating method involves allowing the sterilization condition determination unit 2100 to determine multiple sterilization conditions (S100) and determine whether the multiple sterilization conditions are satisfied simultaneously (S200).

In step S200, a determination is made as to whether all of the multiple sterilization conditions are satisfied at the same time.

In response to determining that all of the multiple sterilization conditions are satisfied at the same time, the sterilization condition determination unit 2100 may be configured to output an operation signal to the sterilization control unit 2200 for sterilization, and the sterilization control unit 2200 may be configured to control the sterilization apparatus 1000 in accordance with predefined settings to ensure that sterilization is performed on each sterilization target area (S300).

Although exemplary embodiment is described as using a plurality of units such as the sterilization condition determination unit 2100 and the sterilization control unit 2200 to perform the exemplary process, it is understood that the exemplary processes may also be performed by one or plurality of modules. Additionally, it is understood that the terms such as controller, control unit, and determination unit refer to one or more hardware devices that include a memory and a processor. The memory is configured to store the modules and the processor is specifically configured to execute said modules to perform one or more processes which are described herein.

Furthermore, control logic of the present disclosure may be embodied as non-transitory computer readable media on a computer readable medium containing executable program instructions executed by a processor, controller/control unit or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

Those skilled in the art to which the present disclosure pertains will understand that the present disclosure can be implemented in various specific forms without changing its technical concept or essential features. Therefore, the embodiments described above are merely exemplary in all aspects and should be understood as non-limiting. The scope of the present disclosure is defined by the claims set forth below, and all modifications or variations derived from the meaning and scope of the claims, as well as the doctrine of equivalents, should be interpreted as being within the scope of the present disclosure.

What is claimed is:

1. A sterilization apparatus comprising:
a housing;
a sterilization unit that irradiates at least one sterilization target area with sterilization light; and
a driving unit that moves the sterilization unit into and out of the housing via an opening provided in the housing and rotates the sterilization unit about a rotation axis.

2. The sterilization apparatus of claim 1, wherein the sterilization unit is moved in a vertical direction through the opening, which is formed on a top surface of the housing.

3. The sterilization apparatus of claim 1, wherein the sterilization unit includes at least one light source to generate the sterilization light and a mounting bracket in which the at least one light source is mounted.

4. The sterilization apparatus of claim 3, wherein the sterilization unit further includes at least one reflector to reflect the sterilization light generated from the at least one light source to the at least one sterilization target area.

5. The sterilization apparatus of claim 4, wherein the at least one reflector includes a plurality of reflective surfaces formed at different locations with respect to the at least one light source.

6. The sterilization apparatus of claim 1, wherein the driving unit includes a first operating part that is coupled to the sterilization unit and allows the sterilization unit to rotate about the rotation axis and a second operating part that allows the sterilization unit to move through the opening of the housing.

7. The sterilization apparatus of claim 6, wherein the second operating part allows the sterilization unit to move in a direction parallel to the rotation axis.

8. The sterilization apparatus of claim 6, wherein the second operating part includes a support bracket that supports the first operating part, and
wherein, as the support bracket moves, the sterilization unit coupled to the first operating part moves through the opening of the housing.

9. The sterilization apparatus of claim 8, wherein the support bracket includes at least one guide rib movably inserted in at least one guide rail, which is formed in the housing.

10. The sterilization apparatus of claim 9, further comprising:
a pressurizing member elastically deformed by the at least one guide rib,
wherein a first end portion of the pressurizing member is coupled to the housing, and a second end portion of the pressurizing member is elastically deformed by the at least one guide rib to exert pressure on one side of the at least one guide rib to allow the at least one guide rib to contact an inner surface of the at least one guide rail.

11. The sterilization apparatus of claim 9, further comprising:
an elastic part formed to extend from the at least one guide rib,
wherein an end portion of the elastic part is elastically deformed by the at least one guide rail.

12. The sterilization apparatus of claim 1, further comprising:
a cover part provided on a side of the sterilization unit.

13. The sterilization apparatus of claim 12, wherein the cover part is formed of a light-transmitting material, and
wherein an optical pattern that reflects or scatters light incident from the outside is formed on at least one surface of the cover part.

14. The sterilization apparatus of claim 1, wherein the sterilization light has a wavelength in a UV-C range.

15. The sterilization apparatus of claim 1, wherein the sterilization light has a wavelength of about 222 nm to about 280 nm.
